(19) United States
(12) Patent Application Publication
(10) Pub. No.: US 2014/0224737 A1
(43) Pub. Date: Aug. 14, 2014

(54) MOLECULARLY IMPRINTED POLYMERS FOR ELIMINATING METABOLITES

(71) Applicant: FRESENIUS MEDICAL CARE DEUTSCHLAND GMBH, BAD HOMBURG (DE)

(72) Inventors: Thomas FICHERT, WARENDORF (DE); INGO BICHLMAIER, HELSINKI (FI)

(73) Assignee: FRESENIUS MEDICAL CARE DEUTSCHLAND GMBH, BAD HOMBURG (DE)

(21) Appl. No.: 14/178,677

(22) Filed: Feb. 12, 2014

Related U.S. Application Data
(63) Continuation of application No. 13/635,245, filed on Sep. 14, 2012, now abandoned.

(30) Foreign Application Priority Data
Mar. 19, 2010 (DE) 10 2010 012 199.1

(51) Int. Cl. A61M 1/16

(52) U.S. Cl. CPC A61M 1/1654
USPC 210/646; 524/612; 524/548

(57) ABSTRACT

The present invention relates to polymers imprinted by retention solutes, use of same as well as compositions containing same.

The present invention relates to polymers imprinted by retention solutes, use of same as well as compositions containing same.

In dialysis treatment, in particular in peritoneal dialysis treatment, osmotic agents, which serve to remove excess water from the dialysis patient, are used.

The peritoneal dialysis method is based on the fact that a solution containing osmotically active compounds is introduced through a catheter into the abdominal cavity of the dialysis patient. This solution is left in the patient's abdominal cavity for a certain period of time (usually a few hours) and manifests its osmotic effect there; in other words, endogenous water is withdrawn from the patient into the abdominal cavity. After a certain dwell time, the peritoneal dialysis solution, which is then dilute, is removed through a catheter.

This principle is employed in various methods of peritoneal dialysis treatment. For example, the methods of intermittent peritoneal dialysis (IPD), nocturnal intermittent peritoneal dialysis (NIPD), continuous cyclic peritoneal dialysis (CCPD) or continuous ambulant peritoneal dialysis (CAPD) may be used as needed. Devices which support the patient in performing the peritoneal dialysis method are used in IPD, NIPD and CCPD. CAPD is a manual method.

Adding osmotically active compounds should ensure in particular that the osmotic pressure of the peritoneal dialysis solution is high enough during the entire dwell time in the abdominal cavity to withdraw water from the patient; i.e., water goes from the patient's circulation into the abdominal cavity (ultrafiltration).

In addition to the secretion of water, another important function of the kidneys is to eliminate metabolites (renal elimination).

However, the elimination of metabolites, which are eliminated exclusively or predominantly renally, is disturbed in patients with restricted renal function, resulting in retention of these dissolved or protein-bound metabolites in the patient's body. These metabolites that are not eliminated are also referred to as “uremic retention solutes” (cf. Vanholder et al., Kidney International, 63 (2003), 1934-1943).

It is known that these uremic retention solutes can lead to pathophysiological signs and symptoms in a renally insufficient patient (Uremic Syndrome; cf. Vanholder et al., Hemodialysis International, 7 (2003), 156-161).

The uremic retention solutes, which lead to pathophysiological signs and symptoms, are also referred to as uremic toxins. For the health of the dialysis patient, it is important for the uremic retention solutes to be eliminated from the body.

In dialysis treatment, excess endogenous water is withdrawn from the dialysis patient. Metabolites are also nonselectively eliminated from the patient's endogenous water into the dialysis solution along the concentration gradient. However, this elimination may be insufficient. There may thus be some retention of metabolites even in dialysis patients, which may in turn contribute to the uremic syndrome.

The synthesis of molecularly imprinted polymers that bind creatinine is described in the literature (Tsai and Syu, Biomaterials, 26 (2005), 2759-2766; Hsieh et al., Biomaterials, 27 (2006), 2083-2089).

The object of the present invention is to increase the elimination of uremic retention solutes during dialysis treatment.

This object is achieved by the subject matter of the patent claims.

It has been found that the use of polymers that are imprinted by uremic retention solutes increases the elimination of metabolites in dialysis treatment. This is a result of the increased specific binding of the retention solute to the imprinted polymer. There is thus increased elimination of the uremic retention solute.

Furthermore, these molecularly imprinted polymers also have a high osmotic efficacy, so they may be used as osmotics in dialysis solutions.

The inventive, molecularly imprinted polymers also have a dual mechanism of action: first, they bind certain uremic retention solutes and thus increase their elimination (specific elimination) in dialysis treatment; secondly, they have an osmotic activity themselves and thus support the removal of water and the nonspecific elimination of metabolites along the concentration gradient associated with this removal of water.

A first subject matter of this invention relates to a molecularly imprinted polymer, which is imprinted by at least one uremic retention solute, for use in dialysis treatment.

In the sense of this description, the term “molecularly imprinted polymer” stands for a polymer that is synthesized by polymerizing or crosslinking monomers in the presence of a template molecule (“template”). At least one uremic retention solute is used here as the template molecule.

For the purposes of this description, the term “monomer” refers to a compound that can be polymerized and/or crosslinked (cf. also: Pure and Applied Chemistry, 68 (1996), 2287). The person skilled in the art will know of monomers that can be used for polymerization.

In the sense of this description, monomers may be compounds which themselves consist of only a single compound, but the term also includes compounds that have been dimerized, oligomerized or polymerized from more than one single compound, such as dimers, oligomers or polymers.

In the sense of this description, the term “dimer” comprises a compound that has been produced from two individual compounds by dimerization. In the sense of this description, the term “oligomer” comprises a compound that has been produced from three to nine individual compounds by polymerization (oligomerization). In the sense of this description, the term “polymer” stands for a macromolecular compound that has been produced from ≧10 individual compounds by polymerization.

In a preferred embodiment, the inventive, molecularly imprinted polymer imprinted by at least one uremic retention solute, which is selected from the group consisting of 1-methyladenosine, 1-methylguanosine, 1-methylinosine, asymmetrical dimethylarginine, α-keto-δ-guanidinovaleric acid, α-N-acetylarginine, arab(in)itol, arginic acid, benzyl alcohol, β-guanidinopropionic acid, β-lipotropin, creatine, creatinine, cytidine, dimethylglycine, erythritol, γ-guanidinobutyric acid, guanidine, guanidinoacetic acid, guanidonosuccinic acid, hypoxanthine, malondialdehyde, mannitol, methylguanidine, myoinositol, N²,N²-dimethylguanosine, N⁴-acetylcytidine, N⁶-methyladenosine, N⁶-threonylcarbamoyladenosine, orotic acid, orotidine, oxalate, phenylacetylglutamine, pseudouridine, symmetrical dimethylarginine, sorbitol, taurocyamine, threitol, thymine, uracil, urea, uric acid, uridine, xanthine, xanthosine, 2-methoxyresorcinol, 3-deoxyglucosone, 3-carboxy-4-methyl-5-propyl-2-furanpropionic acid, fructose-lysine, glyoxal, hippuric acid, homocysteine, hydroquinone, indole-3-acetic acid, indoxyl sulfate, kynurenine, kynurenic acid, leptin, melatonin, methylglyoxal, N^{ε}-(carboxymethyl)lysine, p-cresol, pentosidine, phenol, p-hydroxyhippuric acid, putrescine, quinolinic acid, retinal-binding protein, spermidine, spermine, adrenomedullin, atrial natriuretic peptide, β₂-microglobulin, β-endorphin, cholecystokinin, Clara cell protein (CC16), complement factor D, cystatin C, degranulation-inhibiting protein, delta-sleep-inducing peptide, endothelin, hyaluronic acid, interleukin-1β, interleukin-6, κ-Ig light chain, λ-Ig light chain, leptin, methionine-enkephalin, neuropeptide Y, parathyroid hormone, retinol-binding protein, tumor necrosis factor-α, 1-alkyl-2-formyl-3.4-glycosyl-pyrrole, 2-(2-fuoryl)-4(5)-(2-furanyl)-1H-imidazole, 3-deoxyfructosone, 3-hydroxykynurenine, 4-hydroxynonenal, AOPP (advanced oxidation protein products), advanced glycation end products β₂-microglobulin, anthranilic acid, β₂-microglobulin fragments, cadaverine, crossline, dimethylamine, guanosine, imidazolone, malonaldehyde, malondialdehyde, methylamine, N^{ε}-carboxyethyllysine, organic chloramines, oxidized low-density lipoprotein (oxLDL), parathyroid hormone fragments, pyrraline, pyrrole aldehyde, and trimethylamine.

The inventive, molecularly imprinted polymer preferably has an imprinted ratio of ≧1.10.

For the purposes of this description, the term “imprinted ratio” stands for the ratio of the binding capacity of the molecularly imprinted polymer to the binding capacity of the non-molecularly imprinted polymer, where the non-molecularly imprinted polymer has been produced under the same conditions as the molecularly imprinted polymer, the difference being that no template molecule (“template”) has been used.

In a preferred embodiment, the inventive, molecularly imprinted polymer has an imprinted ratio of ≧1.5, more preferably of ≧2, even more preferably of ≧2.5, most preferably of ≧3 and in particular of ≧4.

In another preferred embodiment, the inventive, molecularly imprinted polymer has an imprinted ratio of ≧1.75, more preferably of ≧2.25, even more preferably of ≧2.75, most preferably of ≧3.5 and in particular of ≧5.

The average molecular weight of the inventive polymer is preferably 2000 to 30000 g/mol, more preferably 2500 to 26000 g/mol, even more preferably 3000 to 22000 g/mol, even more preferably 3500 to 20000 g/mol, most preferably 4000 to 18000 g/mol and in particular 5000 to 15000 g/mol.

In another preferred embodiment, the average molecular weight of the inventive polymer is 15000 to 25000 g/mol, in particular 18000 to 22000 g/mol.

The inventive polymer preferably has an average degree of polymerization of 10 to 170, more preferably of 11 to 130, even more preferably of 12 to 100, most preferably of 13 to 80 and in particular of 14 to 50.

In another preferred embodiment, the average degree of polymerization of the inventive, molecularly imprinted polymer is 80 to 140, more preferably 85 to 135, even more preferably 90 to 130, most preferably 95 to 125 and in particular 100 to 120.

A 7.5 wt % aqueous solution of the inventive, molecularly imprinted polymer preferably has a theoretical osmolarity of >5 mosm/L, more preferably of mosm/L, even more preferably of ≧10.0 mosm/L, most preferably of ≧12.5 mosm/L and in particular of ≧15 mosm/L.

For the purpose of this description, the term “theoretical osmolarity” stands for the osmolarity calculated theoretically. A person skilled in the art will know of methods of calculating this value.

In a preferred embodiment, the colloid-osmotic pressure of a 7.5 wt % solution of the inventive, molecularly imprinted polymer is ≧50 mosm/L or ≧60 mosm/L, more preferably ≧70 mosm/L or ≧80 mosm/L, even more preferably ≧90 mosm/L or ≧100 mosm/L, most preferably ≧110 mosm/L or ≧120 mosm/L and in particular ≧130 mosm/L or ≧140 mosm/L.

In another preferred embodiment, the colloid-osmotic pressure of a 7.5 wt % solution of the inventive polymer is ≧150 mosm/L or ≧160 mosm/L, more preferably ≧170 mosm/L or ≧180 mosm/L, even more preferably ≧190 mosm/L or ≧200 mosm/L, most preferably ≧210 mosm/L or ≧220 mosm/L and in particular ≧230 mosm/L or ≧240 mosm/L.

In another preferred embodiment, the colloid-osmotic pressure of a 7.5 wt % solution of the inventive polymer is 50 to 500 mosm/L, more preferably 75 mosm/L to 400 mosm/L, even more preferably 100 to 300 mosm/L, most preferably 110 mosm/L to 275 mosm/L and in particular 120 mosm/L to 250 mosm/L.

In another preferred embodiment, the colloid-osmotic pressure of a 7.5 wt % solution of the inventive polymer is 100 to 500 mosm/L, more preferably 100 mosm/L to 400 mosm/L, even more preferably 100 to 350 mosm/L, most preferably 100 mosm/L to 325 mosm/L and in particular 100 mosm/L to 290 mosm/L.

For the purpose of this description, the term “colloid-osmotic pressure” stands for the osmotic pressure of the solution, which is measured experimentally and is made up of the osmotic pressure and the oncotic pressure. The person skilled in the art will know of suitable methods of experimental determination of this value.

The osmolality of a 7.5 wt % aqueous solution of the inventive polymer is preferably ≧5 mosm/kg, more preferably ≧7.5 mosm/kg, even more preferably ≧10 mosm/kg, most preferably ≧12.5 mosm/kg and in particular ≧15 mosm/kg.

For the purpose of this description, the term “osmolality” stands for the osmolality of the solution determined experimentally by means of the reduction in freezing point. The person skilled in the art is familiar with methods of determining the reduction in freezing point.

The water solubility of the inventive, molecularly imprinted polymer is preferably ≧5 g/L, more preferably ≧10 g/L, even more preferably ≧20 most preferably ≧50 g/L and in particular ≧75 g/L.

The inventive, molecularly imprinted polymer may even be substituted with various radicals. The inventive polymer is preferably substituted with cationic, anionic, deprotonizable or protonizable radicals that increase the osmotic efficacy of the polymer. Preferred deprotonizable and/or anionic radicals are derived from dicarboxylic acids or tricarboxylic acids in particular.

Suitable dicarboxylic acids are selected from the group comprising, for example, oxalic acid, oxalacetic acid, ketoglutaric acid, glutamic acid, aspartic acid, fumaric acid, maleic acid, malic acid, malonic acid, succinic acid, glutaric acid, adipic acid, pimelic acid, suberic acid, azelaic acid, sebacic acid. In another preferred embodiment, the dicarboxylic acid is oxalic acid, glutamic acid, aspartic acid, maleic acid or succinic acid. Maleic acid and succinic acid are preferred in particular.

Suitable tricarboxylic acids preferably include citric acid or isocitric acid, in particular citric acid.

The inventive polymer may be esterified with dicarboxylic acids and/or tricarboxylic acids. The inventive polymer has a degree of substitution of 0.01 to 3, more preferably of 0.05 to 2.5, even more preferably of 0.1 to 2, most preferably of 0.25 to 1.5 and in particular of 0.5 to 1.

In another preferred embodiment, the inventive polymer has a degree of substitution of 0.02±0.01 or 0.05±0.025, more preferably of 0.1±0.05, even more preferably of 0.5±0.25, most preferably of 1±0.5 and in particular of 1.5±0.75.

In an especially preferred embodiment, the inventive polymer has a degree of substitution of 0.02±0.005 or 0.05±0.0125, more preferably of 0.1±0.025, even more preferably of 0.5±0.125, most preferably of 1±0.25 and in particular of 1.5±0.375.

The molecularly imprinted polymer is preferably derived from a) at least one monomer or b) at least one monomer and at least one crosslinking agent.

The monomer is preferably selected from the group consisting of the saccharides, the amino acids, peptides or olefinic monomers.

Preferred saccharides are monomers such as glucose, fructose, arabinose, xylose, galactose, mannose, N-acetylglucosamine or glucosamine as well as dimers, oligomers or polymers, constructed from the aforementioned saccharide monomers. Preferred in particular are the monomers glucose and fructose as well as dimers, oligomers and polymers constructed from glucose and/or fructose. The oligosaccharides may also be cyclic, such as, for example, α-, β- or γ-cyclodextrin. Preferred polymers include inulin or starch as well as their derivatives, preferably degraded starch (starch hydrolysate; hydrolyzed polydextrin).

Preferred amino acids are selected from the group comprising alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Preferred peptides are dimers, oligomers or polymers, constructed from amino acids selected from the group given above.

The olefinic monomers are preferably any compound containing at least one C—C double bond that is accessible for polymerization. The person skilled in the art will know of such monomers. Preferred olefinic monomers include 4-vinylpyridine and divinyl benzene.

The crosslinking agent is preferably selected from the group comprising glyoxal, 1,2-diahaloethane, 1,3-diahalopropane, halocarboxylic acid halide, epichlorohydrin, 4-chloro-1,2-epoxybutane, 1,2,3,4-diepoxybutane, tetramethylene diisocyanate and hexamethylene diisocyanate.

The inventive polymer is also suitable as an osmotic agent for adjusting the tonicity of pharmaceutical drugs, in particular drug solutions for parenteral administration.

In a preferred embodiment, the inventive polymer is used in dialysis treatment, preferably in hemodialysis and/or peritoneal dialysis treatment.

The inventive polymer is suitable in particular for use in of peritoneal dialysis treatment.

Another subject matter of this invention relates to a molecularly imprinted polymer obtainable by a method, comprising the steps:
a) Mixing at least one monomer with at least one uremic retention solute;
b) Polymerizing and/or crosslinking the monomers; for use in dialysis treatment.

The monomers, the uremic retention solutes and the crosslinking agent are as defined above.

Another subject matter of this invention relates to a method for the production of the inventive, molecularly imprinted polymer, comprising the steps:
a. Mixing at least one monomer with at least one uremic retention solute selected from the group comprising: 1-methyladenosine, 1-methylguanosine, 1-methylinosine, asymmetrical dimethylarginine, α-keto-δ-guanidinovaleric acid, α-N-acetylarginine, arab(in)itol, arginic acid, benzyl alcohol, β-guanidinopropionic acid, β-lipotropin, creatine, cytidine, dimethylglycine, erythritol, γ-guanidinobutyric acid, guanidine, guanidinoacetic acid, guanidonosuccinic acid, hypoxanthine, malondialdehyde, mannitol, methylguanidine, myoinositol, N²,N²-dimethylguanosine, N⁴-acetylcytidine, N⁶-methyladenosine, N⁶-threonylcarbamoyladenosine, orotic acid, orotidine, oxalate, phenylacetylglutamine, pseudouridine, symmetrical dimethylarginine, sorbitol, taurocyamine, threitol, thymine, uracil, uric acid, uridine, xanthine, xanthosine, 2-methoxyresorcinol, 3-deoxyglucosone, 3-carboxy-4-methyl-5-propyl-2-furanpropionic acid, fructose-lysine, glyoxal, hippuric acid, homocysteine, hydroquinone, indole-3-acetic acid, indoxylsulfate, kynurenine, kynurenic acid, leptin, melatonin, methylglyoxal, N^{ε}-(carboxymethyl)lysine, p-cresol, pentosidine, phenol, p-hydroxyhippuric acid, putrescine, quinolinic acid retinol-binding protein, spermidine, spermine, adrenomedullin, atrial natriuretic peptide, β₂-microglobulin, β-endorphin, cholecystokinin, Clara cell protein (CC16), complement factor D, cystatin C, degranulation-inhibiting protein, delta-sleep-inducing peptide, endothelin, hyaluronic acid, interleukin-1β, interleukin-6, κ-Ig light chain, λ-Ig light chain, leptin, methionine-enkephalin, neuropeptide Y, parathyroid hormone, retinol-binding protein, tumor necrosis factor-α, 1-alkyl-2-formyl-3.4-glycosyl-pyrrole, 2-(2-fuoryl)-4(5)-(2-furanyl)-1H-imidazole, 3-deoxyfructosone, 3-hydroxykynurenine, 4-hydroxynonenal, AOPP (advanced oxidation protein products), advanced glycation end products β₂-microglobulin, anthranilic acid, β₂-microglobulin fragments, cadaverine, crossline, dimethylamine, guanosine, imidazolone, malonaldehyde, malondialdehyde, methylamine, N^{ε}-carboxyethyllysine, organic chloramines, oxidized low-density lipoprotein (oxLDL), parathyroid hormone fragments, pyrraline, pyrrole aldehyde, and trimethylamine; and
b. Initiating the polymerization and/or crosslinking reaction.

The polymerization reaction can begin already during the mixing of the components or may be initiated by adding suitable polymerization initiators, such as AIBN, for example. The person skilled in the art is familiar with polymerization initiators.

For example, it is also possible to initiate the polymerization by raising the temperature and/or by the action of electromagnetic radiation.

The crosslinking reaction is preferably initiated by adding a crosslinking agent, Suitable crosslinking agents are as defined above.

Another subject matter of this invention relates to dialysis solutions containing at least one inventive, molecularly imprinted polymer.

In a preferred embodiment, the inventive dialysis solution is a hemodialysis solution or a peritoneal dialysis solution. The inventive dialysis solution is a peritoneal dialysis solution in particular.

Dosage forms that are used in dialysis treatment are preferably concentrates in multicomponent systems or ready-to-use dialysis solutions.

For the purposes of this invention, the term “dialysis solution” comprises a ready-to-use dosage form for dialysis treatment, i.e., a liquid preparation, which is suitable for administration as such. In particular, the dialysis solution need not be diluted or mixed with other preparations prior to administration.

In contrast with the dialysis solutions described above, concentrates, which may be in liquid, solid or semisolid form, are diluted with water or aqueous solutions or dissolved in water or aqueous solutions prior to administration. Similarly, the components of a multicomponent system must be mixed together prior to administration to obtain a ready-to-use dialysis solution. Concentrates and multicomponent systems may be regarded as a precursors of the inventive dialysis solution.

The inventive dialysis solution is preferably a hemodialysis solution or a peritoneal dialysis solution. Hemodialysis and peritoneal dialysis solutions usually contain electrolytes in a concentration which essentially corresponds to the plasma electrolyte concentration. Electrolytes usually include sodium, potassium, calcium, magnesium and chloride ions.

Dialysis solutions usually have a physiologically tolerable pH. This is preferably achieved through buffers (buffer systems), which may even contribute to the total electrolyte content. The buffers are preferably bicarbonate, lactate or pyruvate.

Furthermore, dialysis solutions usually have a physiologically tolerable osmolarity. This is usually achieved by the electrolytes contained in the dialysis solution and inventive, molecularly imprinted polymers, which are physiologically tolerable as osmotically active compounds (osmotics) in the desired concentration.

The inventive dialysis solution has an osmolarity in the range of preferably 200 to 550 mosm/L.

In the case when the inventive dialysis solution is a hemodialysis solution, the osmolarity is preferably 200 to 350 mosm/L or 210 to 340 mosm/L, more preferably 220 to 330 mosm/L, even more preferably 230 to 320 mosm/L, most preferably 240 to 310 mosm/L and in particular 250 to 300 mosm/L. The person skilled in the art will be familiar with methods of measuring the osmolarity and the osmotic pressure. For example, these values may be determined with the aid of a membrane osmometer or other suitable measurement methods.

In the case when the inventive dialysis solution is a peritoneal dialysis solution, the osmolarity is preferably 200 to 570 mosm/L or 210 to 560 mosm/L, more preferably 220 to 550 mosm/L, even more preferably 230 to 540 mosm/L, most preferably 240 to 530 mosm/L and in particular 250 to 520 mosm/L. In a preferred embodiment, the osmolarity is 250±50 mosm/L or 250±45 mosm/L, more preferably 250±35 mosm/L, even more preferably 250±25 mosm/L, most preferably 250±15 mosm/L and in particular 250±10 mosm/L. In another preferred embodiment, the osmolarity is 300±50 mosm/L or 300±45 mosm/L, more preferably 300±35 mosm/L, even more preferably 300±25 mosm/L, most preferably 300±15 mosm/L and in particular 300±10 mosm/L. In another preferred embodiment, the osmolarity is 350±50 mosm/L or 350±45 mosm/L, more preferably 350±35 mosm/L, even more preferably 350±25 mosm/L, most preferably 350±15 mosm/L and in particular 300±10 mosm/L. In another preferred embodiment, the osmolarity is 400±50 mosm/L or 400±45 mosm/L, more preferably 400±35 mosm/L, even more preferably 400±25 mosm/L, most preferably 400±15 mosm/L and in particular 300±10 mosm/L. In another preferred embodiment, the osmolarity is 450±50 mosm/L or 450±45 mosm/L, more preferably 450±35 mosm/L, even more preferably 450±25 mosm/L, most preferably 450±15 mosm/L and in particular 450±10 mosm/L. In another preferred embodiment, the osmolarity is 500±50 mosm/L or 500±45 mosm/L, more preferably 500±35 mosm/L, even more preferably 500±25 mosm/L, most preferably 500±15 mosm/L and in particular 500±10 mosm/L.

The inventive dialysis solution preferably has a pH of 4.0 to 8.0, more preferably of 4.2 to 7.5, even more preferably of 4.4 to 6.8, most preferably of 4.6 to 6.0 or 4.8 to 5.5 and in particular of 5.0 to 5.2 or 5.0±0.1; measured at room temperature (20 to 23° C.). In a preferred embodiment, the pH is 4.8±1.0 or 4.8±0.8, more preferably 4.8±0.7 or 4.8±0.6, even more preferably 4.8±0.5 or 4.8±0.4, most preferably 4.8±0.3 or 4.8±0.2 and in particular 4.8±0.1. In another preferred embodiment, the pH is 5.0±1.0 or 5.0±0.8, more preferably 5.0±0.7 or 5.0±0.6, even more preferably 5.0±0.5 or 5.0±0.4, most preferably 5.0±0.3 or 5.0±0.2 and in particular 5.0±0.1. In another preferred embodiment, the pH is 5.2±1.0 or 5.2±0.8, more preferably 5.2±0.7 or 5.2±0.6, even more preferably 5.2±0.5 or 5.2±0.4, most preferably 5.2±0.3 or 5.2±0.2 and in particular 5.2±0.1. In another preferred embodiment, the pH is 5.5±1.0 or 5.5±0.8, more preferably 5.5±0.7 or 5.5±0.6, even more preferably 5.5±0.5 or 5.5±0.4, most preferably 5.5±0.3 or 5.5±0.2 and in particular 5.5±0.1. In another preferred embodiment, the pH is 6.0±1.0 or 6.0±0.8, more preferably 6.0±0.7 or 6.0±0.6, even more preferably 6.0±0.5 or 6.0±0.4, most preferably 6.0±0.3 or 6.0±0.2 and in particular 6.0±0.1. In another preferred embodiment, the pH is 6.5±1.0 or 6.5±0.8, more preferably 6.5±0.7 or 6.5±0.6, even more preferably 6.5±0.5 or 6.5±0.4, most preferably 6.5±0.3 or 6.5±0.2 and in particular 6.5±0.1. In another preferred embodiment, the pH is 7.0±1.0 or 7.0±0.8, more preferably 7.0±0.7 or 7.0±0.6, even more preferably 7.0±0.5 or 7.0±0.4, most preferably 7.0±0.3 or 7.0±0.2 and in particular 7.0±0.1. In another preferred embodiment, the pH is 7.4±1.0 or 7.4±0.8, more preferably 7.4±0.7 or 7.4±0.6, even more preferably 7.4±0.5 or 7.4±0.4, most preferably 7.4±0.3 or 7.4±0.2 and in particular 7.4±0.1. In another preferred embodiment, the pH is 8.0±1.0 or 8.0±0.8, more preferably 8.0±0.7 or 8.0±0.6, even more preferably 8.0±0.5 or 8.0±0.4, most preferably 8.0±0.3 or 8.0±0.2 and in particular 8.0±0.1.

The inventive dialysis solution contains one or more (for example, two, three, four or five) inventive, molecularly imprinted polymers; where these are defined as indicated above.

The inventive dialysis solution contains the inventive, molecularly imprinted polymer preferable in a total concentration of 0.001 mM to 10 M or 0.01 to 1.0 M, more preferably 0.10 to 500 mM, even more preferably 1.0 to 250 mM, most preferably 10 to 100 mM and in particular 25 to 90 mM. In a preferred embodiment, the total concentration is 25±24 mM, more preferably 25±20 mM, even more preferably 25±15 mM, most preferably 25±10 mM and in particular 25±5 mM. In another preferred embodiment, the total concentration is 50±25 mM, more preferably 50±20 mM, even more preferably 50±15 mM, most preferably 50±10 mM and in particular 50±5 mM. In another preferred embodiment, the total concentration is 75±25 mM, more preferably 75±20 mM, even more preferably 75±15 mM, most preferably 75±10 mM and in particular 75±5 mM. In another preferred embodiment, the total concentration is 100±25 mM, more preferably 100±20 mM, even more preferably 100±15 mM, most preferably 100±10 mM and in particular 100±5 mM. In another preferred embodiment, the total concentration is 200±25 mM, more preferably 200±20 mM, even more preferably 200±15 mM, most preferably 200±10 mM and in particular 200±5 mM. The total concentration is preferably calculated based on the average molecular weight of the inventive polymer.

The inventive dialysis solution contains the inventive, molecularly imprinted polymer in a total mass concentration of preferably 0.01 g/L to 1.0 kg/L, more preferably of 0.1 to 750 g/L, even more preferably of 1.0 to 500 g/L, most preferably 10 to 250 g/L and in particular of 100 to 200 g/L. In a preferred embodiment, the total mass concentration is 25±24 g/L, more preferably 25±20 g/L, even more preferably 25±15 g/L, most preferably 25±10 g/L and in particular 25±5 g/L. In another preferred embodiment, the total mass concentration is 50±25 g/L, more preferably 50±20 g/L, even more preferably 50±15 g/L, most preferably 50±10 g/L and in particular 50±5 g/L. In another preferred embodiment, the total mass concentration is 75±25 g/L, more preferably 75±20 g/L, even more preferably 75±15 g/L, most preferably 75±10 g/L and in particular 75±5 g/L. In another preferred embodiment, the total mass concentration is 100±25 g/L, more preferably 100±20 g/L, even more preferably 100±15 g/L, most preferably 100±10 g/L and in particular 100±5 g/L. In another preferred embodiment, the total mass concentration is 200±25 g/L, more preferably 200±20 g/L, even more preferably 200±15 g/L, most preferably 200±10 g/L and in particular 200±5 g/L.

The inventive dialysis solution may also contain other osmotically active substances such as, for example, glucose, polyglucose, crosslinked glucose or polyglucose, mannitol or glycerol.

The inventive dialysis solution preferably contains one or more electrolytes.

In the sense of this invention, the term “electrolyte” stands for a substance that contains free ions and has an electric conductivity. The electrolyte preferably dissociates completely into cations and anions without significantly altering the pH of an aqueous composition. This property differentiates electrolytes from buffer substances. The electrolytes are preferably present in a concentration that results in essentially complete dissociation in water.

Preferred electrolytes are selected from the group comprising the alkali metals, such as, for example, Na⁺ and K⁺, and the alkaline earth metals, such as, for example, Ca²⁺ and Mg²⁺. A preferred anion is Cl⁻.

The inventive dialysis solution may contain additional anions, such as, for example, bicarbonate, dihydrogen phosphate, hydrogen phosphate, phosphate, acetate, lactate and pyruvate. However, these anions (in suitable combinations with cations) are not identified as electrolytes but instead as buffers based on their buffering capacity in the sense of this invention.

In a preferred embodiment, the inventive dialysis solution contains Na⁺ ions. The concentration of Na⁺ ions is preferably 10 to 200 mM or 50 to 190 mM, more preferably 100 to 180 mM or 110 to 170 mM, even more preferably 115 to 165 mM or 120 to 160 mM, most preferably 125 to 155 mM and in particular 130 to 150 mM. In another preferred embodiment, the inventive dialysis solution does not contain any Na⁺ ions.

In a preferred embodiment, the inventive dialysis solution contains K⁺ ions. The concentration of K⁺ ions is preferably 0.10 to 20 mM, more preferably 0.25 to 15 mM, even more preferably 0.50 to 10 mM, most preferably 0.75 to 7.5 mM and in particular 1.0 to 5.0 mM. In another preferred embodiment, the concentration of K⁺ ions is 1.0±0.75, 2.0±0.75, 3.0±0.75, 4.0±0.75 or 5.0±0.75 mM and in particular 1.0±0.50, 2.0±0.50, 3.0±0.50, 4.0±0.50 or 5.0±0.50. In another preferred embodiment, the inventive dialysis solution does not contain any K⁺ ions.

In a preferred embodiment, the inventive dialysis solution contains Ca²⁺ ions. The concentration of Ca²⁺ ions is preferably 0.1 to 3 mM, more preferably 0.25 to 2.75 mM, even more preferably 0.5 to 2.5 mM, most preferably 0.75 to 2.25 mM and in particular 1 to 2 mM. In another preferred embodiment, the concentration of Ca²⁺ ions is 0.25, 0.5, 0.75, 1, 1.25, 1.5, 1.75 or 2 mM. In another preferred embodiment, the inventive dialysis solution does not contain any Ca²⁺ ions.

In a preferred embodiment, the inventive dialysis solution contains Mg²⁺ ions. The concentration of Mg²⁺ ions is preferably 0.01 to 1 mM, more preferably 0.05 to 0.75 mM, even more preferably 0.1 to 0.5 mM, most preferably 0.15 to 0.4 mM and in particular 0.2 to 0.3 mM. In another preferred embodiment, the concentration of Mg²⁺ ions is 0.05, 0.075, 0.1, 0.2, 0.25, 0.50 or 0.75 mM. In another preferred embodiment, the inventive dialysis solution does not contain any Mg²⁺ ions.

In a preferred embodiment, the inventive dialysis solution contains Cl⁻ ions. The concentration of Cl⁻ ions is preferably 10 to 300 mM, more preferably 25 to 250 mM, even more preferably 50 to 200 mM, most preferably 75 to 150 mM and in particular 80 to 125 mM. In another preferred embodiment, the concentration of Cl⁻ ions is 100±50 mM, more preferably 100±25 mM, most preferably 100±10 mM and in particular 96±4 mM. In another preferred embodiment, the inventive dialysis solution does not contain any Cl⁻ ions.

The inventive dialysis solution preferably contains one or more buffers.

The person skilled in the art will know of suitable buffers. Buffers usually include lactate, bicarbonate, carbonate, dihydrogen phosphate, hydrogen phosphate, phosphate, pyruvate, citrate, isocitrate, succinate, fumarate, acetate and lactate salts. The person skilled in the art will know that the corresponding cation of the anions listed above is a component of the buffer that is used to adjust the pH (e.g., Na^{⊕} as a component of the buffer NaHCO₃). However, if the buffer salt dissociates in water, it will also act like an electrolyte. For the purposes of this description, the concentrations of cations or anions and the total concentration of ions are calculated, regardless of whether they are used as a component of electrolytes, buffers or other compounds (e.g., as a salt of the inventive polymers).

In a preferred embodiment, the buffer contains bicarbonates. Bicarbonate is a buffer system that is tolerated well; in an alkaline medium, it is in equilibrium with carbonate, and in an acidic medium, it is in equilibrium with H₂CO₃ and/or CO₂. In addition to bicarbonates, other buffer systems may also be used, if they have a buffering effect in the range of pH 4 to pH 8, more preferably in the range of pH 5 to pH 7.6 and in particular in the range of pH 7.6, 7.4, 7.2 and/or 7.0; for example, this includes compounds that can be metabolized to bicarbonate in the body, such as lactate or pyruvate.

In another preferred embodiment, the buffer contains the salt of a weak acid, preferably lactate. The acid strength (pKₛ) of the weak acid is preferably ≦55. The buffer may also be a mixture of substances having a buffering action, e.g., a mixture containing bicarbonate and a salt of a weak acid (e.g., lactate). A low bicarbonate concentration has the advantage that the CO₂ pressure in the container is low.

In a preferred embodiment, the inventive dialysis solution is buffered by bicarbonate. The bicarbonate concentration is preferably 1.0 to 200 mM, more preferably 2.5 to 150 mM, even more preferably 5 to 100 mM, most preferably 5 to 75 mM or 10 to 50 mM and in particular 20 to 30 mM. In another preferred embodiment, the bicarbonate concentration is 25 mM. In another preferred embodiment, the inventive dialysis solution does not contain any bicarbonate.

In a preferred embodiment, the inventive dialysis solution is buffered by lactate. The lactate concentration is preferably 1.0 to 200 mM, more preferably 2.5 to 150 mM, even more preferably 5 to 100 mM, most preferably 10 to 50 mM or 10 to 25 mM and in particular 15 mM. In another preferred embodiment, the inventive dialysis solution does not contain any lactate.

In a preferred embodiment, the inventive dialysis solution is buffered by acetate. Die acetate concentration is preferably 1.0 to 100 mM, more preferably 1.0 to 50 mM, even more preferably 1.0 to 25 mM, most preferably 1.0 to 10 mM or 2.0 to 7.5 mM and in particular 2.5 to 7.0 mM. In another preferred embodiment, the inventive dialysis solution does not contain any acetate.

The total volume of dialysis solution is not limited. The volume is usually several liters (suitable dosage form for administration to one patient) to a few hundred liters (suitable supply volume for more than one patient).

As already explained above, the term “dialysis solution” is to be understood in the sense of this invention to refer to a ready-to-use dialysis solution, i.e., the dialysis solution may be used directly for dialysis treatment (hemodialysis or peritoneal dialysis).

In a preferred embodiment, the inventive dialysis solution is a peritoneal dialysis solution as described below.

The peritoneal dialysis solution is tailored biochemically so that it essentially corrects the metabolic acidosis associated with renal failure. The peritoneal dialysis solution preferably contains bicarbonate in approximately physiological concentrations. In a preferred embodiment, the peritoneal dialysis solution contains bicarbonate in a concentration of approx. 20 to 30 mM. In another preferred embodiment, the peritoneal dialysis solution contains a bicarbonate concentration of 25 mM.

Furthermore, the peritoneal dialysis solution preferably contains carbon dioxide with a partial pressure (pCO₂) of less than 60 mmHg. In a preferred embodiment, the pCO₂ of the peritoneal dialysis solution is essentially the same as the pCO₂, measured in blood vessels.

Furthermore, the peritoneal dialysis solution preferably has a pH of approx. 7.4. Therefore, the peritoneal dialysis solution is a physiologically tolerable solution.

The peritoneal dialysis solution preferably contains a weak acid with a pKₛ≦5.5. The weak acids are preferably compounds that occur as physiological metabolites in the glucose metabolism. The weak acid is preferably selected from the group comprising lactate, pyruvate, citrate, isocitrate, ketoglutarate, succinate, fumarate, malate and oxaloacetate. These acids may be present in the peritoneal dialysis solution either alone or as a mixture. The weak acids are preferably present in a concentration of 10 to 20 meq/L and essentially as sodium salts in the peritoneal dialysis solution. The weak acid is preferably present in the peritoneal dialysis solution in an amount corresponding to a daily metabolic hydrogen production of approx. 1 meq/kg.

The peritoneal dialysis solution contains at least one inventive, molecularly imprinted polymer as defined above.

The inventive peritoneal dialysis solution preferably contains a concentration of bicarbonate and has a pCO₂, similar to that measured in healthy patients that are not in renal failure. The weak acid diffuses along the concentration gradient of the dialysis solution into the blood of the dialysis patient and thus corrects the metabolic acidosis of the dialysis patient.

Another subject matter of this invention relates to multicomponent systems for producing the ready-to-use dialysis solutions. The solutions are preferably prepared by a method which is described in detail, i.e., by following appropriate instructions (protocol). Said preparation may be performed manually, e.g., by mixing individual components or by diluting one component with water. However, the solutions may also be prepared by an automated method, e.g., by means of a device that is suitable for this process and may be available commercially. The preparation process need not necessarily lead to a dialysis solution having a static (unchanging) composition, but instead it may also lead to a dialysis solution having a composition that changes continuously, such that this change can be monitored by a suitable device. For example, the inventive, molecularly imprinted polymer may be contained in a dialysis solution, which is diluted continuously during a dialysis treatment, so that the patient is exposed to a declining polymer concentration.

In a preferred embodiment, the inventive dialysis solutions are suitable for use in the treatment of renal insufficiency.

In another preferred embodiment, the inventive dialysis solutions are suitable for use in dialysis treatment.

In another preferred embodiment, the inventive dialysis solutions are suitable for use in hemodialysis and/or peritoneal dialysis treatment.

Another subject matter of this invention relates to a kit that has been configured for the preparation of the inventive dialysis solution, such that the kit comprises:
a first component,
a second component and
optionally one or more additional components,
   and
   the inventive dialysis solution is prepared by mixing the first component with the second component and optionally the additional component(s).

The kit comprises at least one first component and one second component. The kit may also comprise additional components, e.g., a third component and a fourth component. The kit preferably consists of two components, which are preferably different from one another.

In the sense of this invention, the term “component” refers to liquid, semisolid or solid compositions, which may be the same as or different from one another, such that the inventive ready-to-use dialysis solution is obtained by mixing all the components of the kit. A single component preferably contains a portion of the ingredients present in the ready-to-use dialysis solution.

The first component and the second component, independently of one another, may be solid, semisolid or liquid. In the case when the components are liquid, they may be solutions or dispersions (e.g., dispersions or suspensions).

In a preferred embodiment, the first component is liquid, preferably pure water or an aqueous solution, and the second component is also liquid. In another preferred embodiment, the first component is liquid, preferably pure water or an aqueous solution, and the second component is solid, preferably a powdered mixture.

The first component is preferably a solution, containing osmotically active substances (e.g., inventive polymer), calcium ions, magnesium ions, hydronium ions and chloride ions.

The inventive kit may be designed in various ways. For example, the individual components may be present in separate containers (e.g., individual bags). However, the inventive kit is preferably a multichamber container system (e.g., a flexible or rigid multichamber container system), preferably a flexible multichamber bag system.

The inventive kit is preferably a multichamber container system, which contains the first component, the second component and optionally one or more additional components in chambers that are separated from one another by soluble and/or breakable separation systems (e.g., breakable separation parts), wherein the first component, the second component and optionally the one or more additional components can be mixed with one another after dissolving and/or breaking the separation system to obtain the inventive dialysis solution.

The multichamber container may be a multichamber plastic bag, containing a separate chamber for each individual component. The container preferably contains the individual component solutions in chambers, each being separated from the others by separation elements.

The multichamber container is preferably a two-chamber bag having a first chamber and a second chamber, wherein the chambers are separated from one another by a soluble and/or breakable separation system, and the first chamber contains the first component, and the second chamber contains the second component. Dissolving and/or breaking of the separation results in mixing of the two components and yields the ready-to-use dialysis solution. The first chamber and the second chamber are preferably arranged adjacently in the container and are separated from one another by the separation system. The separation system is preferably a separation seam (e.g., a soluble or breakable weld). The separation seam preferably opens by applying a pressure to one of the chambers, whereupon the separation seam breaks and/or dissolves and the contents of the two chambers are mixed and the mixture can be used as a ready-to-use dialysis solution in dialysis treatment.

The first component of the inventive kit is preferably a sterile solution, which contains an acid and has a pH of ≦6.0; the second component is preferably likewise a sterile solution, which preferably contains a buffer and has a pH of ≧7.0.

The inventive polymer may be present in the same or different concentrations in the first component or in the second component as well as in both components. In a preferred embodiment, the inventive polymer is contained only in the first (acidic) component. In another preferred embodiment, the inventive polymer is contained only in the second (basic) component. The first component and/or the second component and/or the optional additional component(s) may contain one or more electrolytes or also buffers.

The person skilled in the art will know that mixing the individual components usually involves a dilution effect for the case when the components contain the ingredients in different concentrations. For example, if the inventive polymer is contained only in one of the components, mixing of this component with at least one other component leads to an increase in volume with respect to the amount of the inventive polymer present and thus to a dilution, i.e., a reduction in the polymer concentration. Consequently, the component preferably contains the inventive polymer in a higher concentration than the ready-to-use dialysis solution.

The concentration of inventive polymer in the component is preferably close to the saturation concentration at a temperature of 5° C. to ensure an adequate stability in storage at elevated temperatures.

In a preferred embodiment, the total mass concentration of an inventive polymer in the component is 0.01 g/L to 1.0 kg/L, more preferably 0.1 to 750 g/L, even more preferably 1.0 to 500 g/L, most preferably 10 to 250 g/L and in particular 100 to 200 g/L. In another preferred embodiment, the total mass concentration of an inventive polymer in the component is 25±24 g/L, more preferably 25±20 g/L, even more preferably 25±15 g/L, most preferably 25±10 g/L and in particular 25±5 g/L. In another preferred embodiment, the total mass concentration of an inventive polymer in the component is 50±25 g/L, more preferably 50±20 g/L, even more preferably 50±15 g/L, most preferably 50±10 g/L and in particular 50±5 g/L. In another preferred embodiment, the total mass concentration of an inventive polymer in the component is 75±25 g/L, more preferably 75±20 g/L, even more preferably 75±15 g/L, most preferably 75±10 g/L and in particular 75±5 g/L. In another preferred embodiment, the total mass concentration of an inventive polymer in the component is 100±25 g/L, more preferably 100±20 g/L, even more preferably 100±15 g/L, most preferably 100±10 g/L and in particular 100±5 g/L, In another preferred embodiment, the total mass concentration of an inventive polymer in the component is 200±25 g/L, more preferably 200±20 g/L, even more preferably 200±15 g/L, most preferably 200±10 g/L and in particular 200±5 g/L.

In a preferred embodiment, the second component contains the total amount of inventive molecularly imprinted polymer and a suitable buffer that adjusts the pH of the second component to more than 7.0, more preferably to more than 7.5, even more preferably to more than 8.0, most preferably to more than 8.5 and in particular to more than 9.0. This can preferably be achieved by bicarbonate, which may be present, for example, in the form of dissociated sodium bicarbonate and or potassium bicarbonate. In another preferred embodiment, the second component is solid and includes a powdered mixture containing at least one inventive polymer and at least one buffer, e.g., sodium and/or potassium bicarbonate.

The multichamber bag is preferably suitable for preparing a dialysis solution that may be used for peritoneal dialysis treatment and contains the following ingredients, preferably in the following concentrations:
Ca^{2⊕} 0.5 to 5 meq/L:
Mg^{2⊕} 0 to 3.0 meq/L;
Cl^{⊖} 90.5 to 121 meq/L;
K^{⊕} 0 to 4.0 meq/L;
HCO₃^{⊖} 25 to 40 meq/L; wherein

One chamber of the multichamber bag system contains a first acidic concentrate and one other chamber contains a second basic concentrate; wherein the acidic concentrate contains Ca^{2⊕} ions and the basic concentrate contains HCO₃^{⊖} ions but no Ca^{2⊕} ions; and the two concentrates can be mixed with one another after dissolving and/or breaking the separation system (e.g., separating seam); wherein mixing of the two concentrates leads to preparation of the ready-to-use dialysis solution and the pH of the ready-to-use dialysis solution is 7.0 to 7.6.

The basic concentrate preferably contains at least one inventive polymer and optionally glucose and/or polyglucose, whereas the acid concentrate does not contain any inventive polymer or any glucose and/or polyglucose.

The basic concentrate preferably contains an amount of bicarbonate that results in a bicarbonate concentration of the ready-to-use dialysis solution of at least 20 mM. The bicarbonate concentration of the basic component is preferably so high that the ready-to-use dialysis solution has a bicarbonate concentration of 25 mM.

The pH of the basic buffered second concentrate is preferably adjusted by using hydrochloric acid.

The two concentrates are preferably mixed together in a in a volume ratio of 10:1 to 1:10 or 8:1 to 1:8, more preferably 5:1 to 1:5 or 3:1 to 1:3, even more preferably 2:1 to 1:2 and in particular 1:1.

The multichamber bag preferably has a gas barrier film, which prevents the gaseous CO₂ from escaping from the system. The person skilled in the art will know of gas barrier films.

A preferred subject matter of this invention relates to a method for the production of a dialysis solution, wherein the desired mixing ratio is automatically achieved by a dialysis machine or a peritoneal dialysis cycler.

In a preferred embodiment, the invention relates to a solid composition that is suitable for producing the inventive dialysis solution by dissolving it in a defined volume of a solvent (e.g., water). The solid composition is preferably one of the components described above and is thus a component of the inventive kit.

The solid composition contains the inventive polymer in any solid form, e.g., as a powder, granules, pellets, etc. The inventive polymer may be used as a lyophilisate or may be spray-dried.

The inventive solid composition preferably contains a bicarbonate salt, such as sodium or potassium bicarbonate, for example. The substance quantity ratio of bicarbonate to the inventive polymer in the solid composition is preferably 1:100 to 100:1, more preferably 1:50 to 50:1, even more preferably 1:25 to 25:1, most preferably 1:10 to 10:1 and in particular 1:5 to 5:1.

The defined volume of solvent required to prepare the inventive dialysis solution by dissolving the solid composition is preferably 1.0 to 2000 L. The solvent is preferably purified water, sterilized water or water for injection purposes, which may optionally contain one or more of the electrolytes described above, one or more osmotically active substances (e.g., at least one inventive polymer) and/or one or more of the buffers described above.

Another subject matter of this invention relates to the use of at least one inventive polymer for producing the inventive dialysis solution (hemodialysis solution or peritoneal dialysis solution).

Another subject matter of this invention relates to the use of an inventive kit for producing the inventive dialysis solution (hemodialysis solution or peritoneal dialysis solution).

Another subject matter of this invention relates to the use of an inventive solid composition for producing the inventive dialysis solution (hemodialysis solution or peritoneal dialysis solution).

### EXAMPLES

The molecularly imprinted polymers were synthesized according to Hsieh et al., Biomaterials 27 (2006), 2083-2089 (section 2.2). The substance quantity ratio of monomer to crosslinking agent is 1:10 in all experiments.

The binding capacity of the synthesized polymers was determined as described in Hsieh et al. Biomaterials, 27 (2006), 2083-2089 (section 2.4).

| | | Template | |
|---|---|---|---|
| Example | Monomer | Molecule¹⁾ | Imprinted Ratio |
| 1 | degraded starch²⁾ | — | — |
| 2 | degraded starch²⁾ | Creatinine | >1.10 |
| 3 | degraded starch²⁾ | Creatine | >1.10 |
| 4 | β-Cyclodextrin | — | — |
| 5 | β-Cyclodextrin | Creatinine | >1.10 |
| 6 | β-Cyclodextrin | Hypoxanthine | >1.10 |
| 7 | Inulin³⁾ | — | — |
| 8 | Inulin³⁾ | Creatinine | >1.10 |
| 9 | Inulin³⁾ | Indoxyl sulfate | >1.10 |
| 10⁴⁾ | 4-Vpy and DVB⁵⁾ | — | — |
| 11⁴⁾ | 4-Vpy and DVB⁵⁾ | Creatinine | >1.10 |

¹⁾Template molecule (“template”) = uremic retention solute
²⁾Average molecular weight~6400
³⁾Average molecular weight~5000
⁴⁾Synthesized as described in Hsieh et al., Biomaterials 27 (2006), 2083-2089 (section 2.3)
⁵⁾4-Vpy: 4-vinylpyridine; DVB: divinylbenzene

The elimination of the retention solute creatinine is determined as follows: Exemplary compound 5 (3 g) is dissolved in 10 mL of a 0.9% (m/m) aqueous NaCl solution and introduced into a chamber of a Pfeffer cell. Creatinine (50 mg) is dissolved in 10 mL of a 0.9% (w/w) aqueous NaCl solution and introduced into the other chamber of the Pfeffer cell. The two cells are interconnected by a semipermeable membrane (cellulose membrane, cutoff=1000 Da). After 16 hours, the two solutions of the chambers are extracted with chloroform (extraction three times, 25 mL CHCl₃), which is then dried over sodium sulfate. The dried chloroform phase is evaporated in a Rotary evaporator and the residue is then dried for 24 h in vacuo. The residue is dissolved in 5 mL water and the creatinine concentration is determined analytically by HPLC (high-performance liquid chromatography) (method according to Hsieh et al., Biomaterials 27 (2006), 2083-2089 (section 2.6)).

The experiment is repeated using Comparative Exemplary Compound 4.
The chamber with Exemplary Compound 5 had a ˜5% elevated creatinine concentration in comparison with the chamber with Comparative Exemplary Compound 4.

1-13. (canceled)

14: A solid composition suitable for preparing a dialysis solution and comprising a water-soluble combination of
a) at least one template-imprinted polymer that selectively binds the template, the template-imprinted polymer being a crosslinked polymerized mass imprinted by the template,
wherein the crosslinked polymerized mass is derived from (i) at least one saccharide monomer selected from the group consisting of glucose, fructose, arabinose, xylose, galactose, mannose, N-acetylglucosamine, glucosamine, starch, degraded starch, inulin, α-cyclodextrin, β-cyclodextrin, and γ-cyclodextrin and (ii) at least one crosslinking agent, and
wherein the template is at least one uremic retention solute selected from the group consisting of 1-methyladenosine, 1-methylguanosine, 1-methylinosine, asymmetrical dimethylarginine, α-keto-δ-guanidinovaleric acid, α-N-acetylarginine, arab(in)itol, arginic acid, benzyl alcohol, β-guanidinopropionic acid, β-lipotropin, creatine, cytidine, dimethylglycine, erythritol, γ-guanidinobutyric acid, guanidine, guanidinoacetic acid, guanidonosuccinic acid, hypoxanthine, malondialdehyde, mannitol, methylguanidine, myoinositol, N²,N²-dimethylguanosine, N⁴-acetylcytidine, N⁶-methyladenosine, N⁶-threonylcarbamoyladenosine, orotic acid, orotidine, oxalate, phenylacetylglutamine, pseudouridine, symmetrical dimethylarginine, sorbitol, taurocyamine, threitol, thymine, uracil, urea, uric acid, uridine, xanthine, xanthosine, 2-methoxyresorcinol, 3-deoxyglucosone, 3-carboxy-4-methyl-5-propyl-2-furanpropionic acid, fructose-lysine, glyoxal, hippuric acid, homocysteine, hydroquinone, indole-3-acetic acid, indoxylsulfate, kynurenine, kynurenic acid, leptin, melatonin, methylglyoxal, N^{ε}-(carboxymethyl)lysine, p-cresol, pentosidine, phenol, p-hydroxyhippuric acid, putrescine, quinolinic acid retinol-binding protein, spermidine, spermine, adrenomedullin, atrial natriuretic peptide, β₂-microglobulin, β-endorphin, cholecystokinin, Clara cell protein (CC16), complement factor D, cystatin C, degranulation-inhibiting protein, delta-sleep-inducing peptide, endothelin, hyaluronic acid, interleukin-1β, interleukin-6, κ-Ig light chain, λ-Ig light chain, leptin, methionine-enkephalin, neuropeptide Y, parathyroid hormone, retinol-binding protein, tumor necrosis factor-α, 1-alkyl-2-formyl-3,4-glycosyl-pyrrole, 2-(2-fuoryl)-4(5)-(2-furanyl)-1H-imidazole, 3-deoxyfructosone, 3-hydroxykynurenine, 4-hydroxynonenal, advanced oxidation protein products (AOPP), advanced glycation end products, β₂-microglobulin, anthranilic acid, β-microglobulin fragments, cadaverine, crossline, dimethyl amine, guanosine, imidazolone, malonaldehyde, malondialdehyde, methylamine, N^{ε}-carboxyethyllysine, organic chloramines, oxidized low-density lipoprotein (oxLDL), parathyroid hormone fragments, pyrraline, pyrrole aldehyde, and trimethylamine, and
b) one or more components useful in dialysis treatment.

15: The solid composition according to claim 14, wherein the imprinted ratio is ≧1.10.

16: The solid composition according to claim 14, wherein a 7.5 wt % aqueous solution of the molecularly imprinted polymer has a colloid-osmotic pressure ≧50 mosm/L.

17: The solid composition according to claim 14, wherein the crosslinking agent is selected from the group consisting of glyoxal, 1,2-diahaloethane, 1,3-diahalopropane, halocarboxylic acid halide, epichlorohydrin, 4-chloro-1,2-epoxybutane, 1,2,3,4-diepoxybutane, tetramethylene diisocyanate, and hexamethylene diisocyanate.

18: A dialysis solution comprising the solid composition according to claim 16 in a pharmacologically acceptable solvent.

19: A kit for preparing a dialysis solution comprising
a) the solid composition according to claim 14 and
b) a pharmacologically acceptable solvent.

20: A method comprising dialyzing blood with the dialysis solution according to claim 18
